(19) United States
(12) Patent Application Publication
(10) Pub. No.: US 2004/0094154 A1
(43) Pub. Date: May 20, 2004

(54) Arrangement for passive gas sampling

(75) Inventors: Rolf Castor, Hagersten (SE); Magnus Hallback, Bromma (SE)

Correspondence Address: SCHIFF HARDIN & WAITE, Patent Department, 6600 Sears Tower, 233 South Wacker Drive, Chicago, IL, 60606, US

(73) Assignee: Siemens Elema AB

(21) Appl. No.: 10/686,981

(22) Filed: Oct. 16, 2003

(30) Foreign Application Priority Data
Nov. 20, 2002 (SE) 0203426-2

(51) Int. Cl. A61M 16/00

(52) U.S. Cl. 128/204.22

(57) ABSTRACT

An arrangement for passive gas sampling of a breathing gas in a breathing system has an element disposed in a tube for the passive diversion of a portion of the flow of breathing gas to a measurement chamber.

### BACKGROUND OF THE INVENTION

1. Field of the Invention

The present invention relates to an arrangement for passive gas sampling of the type suitable for sampling a breathing gas.

2. Description of the Prior Art

In breathing systems such as ventilators and anesthetic apparatus the breathing gas is analyzed regularly. This may be done directly in the main supply (with a so-called mainstream-analyzer) or by diverting a gas sample to a measuring chamber (so-called sidestream-analyzer).

The diversion of the gas sample can be done actively by means of a pump or the like or passively, for example by creating a pressure variation between the pressure chamber's inlet and outlet. An example of the latter is described in U.S. Pat. No. 6,450,968.

### SUMMARY OF THE INVENTION

An object of the present invention is to provide an alternative to known arrangements for passive gas sampling.

The above object is achieved in accordance with the invention in an arrangement for passive gas sampling of a breathing gas in a breathing system, having a tube in which breathing gas flows, and an element disposed in said tube for passively diverting a portion of the flow of breathing gas to measurement chamber.

A gas sample may be easily obtained passively using an element that diverts the gas sample from a flow of breathing gas to a measurement chamber. The element can take different forms in order to effectively divert the gas sample and furthermore can be designed to divert a gas sample in one or two flow directions.

The above object also is achieved in accordance with the invention in an arrangement for passive gas sampling of a breathing gas in a breathing system, having a housing element in which a measurement chamber is disposed, and having a first connection for reception of an inspiration flow, a second connection for discharging an expiration flow, and a third connection for discharging the inspiration flow and reception of the expiration flow, the housing containing gas flow paths therein configured so that the first connection and the third connection conduct the received inspiration flow and the expiration flow along a substantially straight path toward the measurement chamber.

This solution builds upon the same principle, but instead of an element that diverts the gas sample, here the actual arrangement is designed to, in principle, force the entire gas flow to pass the measurement chamber.

### DESCRIPTION OF THE DRAWINGS

FIG. 1 shows a first embodiment of an arrangement according to the invention.

FIG. 2 shows an element in the arrangement according to the first embodiment.

FIG. 3 shows a second embodiment of an arrangement according to the invention.

FIG. 4 shows a third embodiment of an arrangement according to the invention.

### DESCRIPTION OF THE PREFERRED EMBODIMENTS

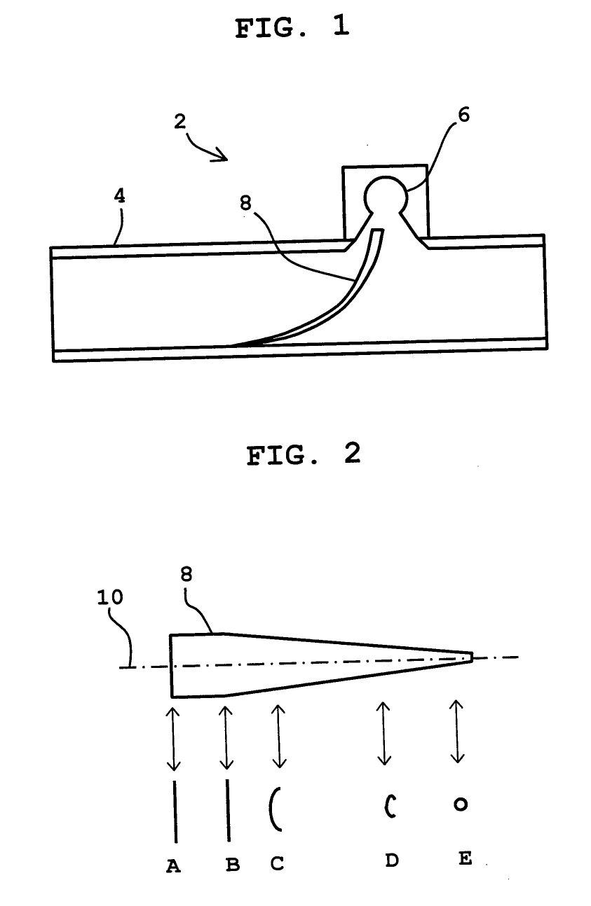

A first embodiment of an arrangement 2 according to the invention is shown in FIG. 1. The arrangement 2 is formed as a tube 4. A gas sample can be analyzed in a measurement chamber 6 according to a known method of analysis, e.g. optical, acoustical or electrochemical.

Gas samples are conducted into the measurement chamber 6 via an element 8 arranged in the tube 4. The element 8 is curved to lead the gas sample into the measurement chamber 6. As is shown in FIG. 2 the element 8 is also curved around a central axis 10. In the lower portion of FIG. 2 the profile is shown at different sections of the element 8. A first section A shows that the element 8 is straight at its base; similarly for the second section B. Then the element 8 is successively curved, as is shown in a third section C and a fourth section D. At its tip the element 8 finally presents an essentially round ring, as is shown in a fifth section E.

The successive curving around the center axis 10 contributes to an optimization of the flow of the gas sample into the measurement chamber 6.

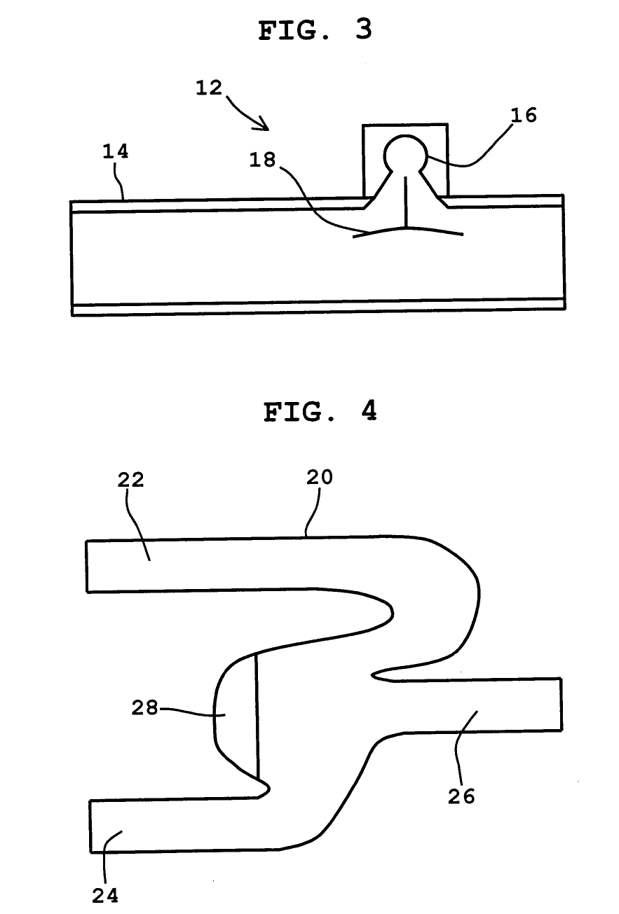

A second embodiment of an arrangement 12 according to the invention is shown in FIG. 3. The arrangement 12 again is formed as a tube 14. A gas sample can be analyzed in a measurement chamber 16 according to a known method of analysis. An element 18 is arranged in the tube 14 to conduct a gas sample into the measurement chamber 16.

Unlike the first exemplary embodiment, the arrangement 12 according to the second exemplary embodiment is designed to sample gas in both flow directions within the tube 14. This can be done by providing the element 18 with an extension in both the directions. The arrangement 12 is therefore suitable for placement near a patient and to sample gas during both inspiration and expiration.

The gas sample will be led in from one side (from the left side in FIG. 3 when the gas flow is from the left to the right and from the right side when the gas flow is from the right to the left) and at the same time the contents of the measurement chamber 16 are forced out through the other side (right to left).

A third embodiment of an arrangement 20 according to the invention is shown in FIG. 4.

The arrangement 20 has a housing element with a first connection 22 for reception of an inspiration flow from a (not shown) breathing arrangement, a second connection 24 for expulsion of an expiration flow and a third connection 26 for expulsion of the inspiration flow and reception of the expiration flow to and from a (not shown) patient. A measurement chamber 28 is formed in the arrangement 20 where a gas sample can be analyzed in a known way. The arrangement 20 is designed so that the first connection 22 and the third connection 26 respectively conduct the received inspiration flow and expiration flow essentially straight towards the measurement chamber 28.

The measurement chamber 28 is essentially completely open to the flow paths in the arrangement 20, which is why it constitutes an intermediate design between a mainstream-analyzer and a sidestream-analyzer. Gas samples are efficiently swapped during both inspiration and expiration as the breathing gas flow is then directed toward the measurement chamber 28.

Combinations of the described exemplary embodiments can easily be done.

For example the design of the element 8 in FIG. 1 can be used for the element 18 in FIG. 3.

Although modifications and changes may be suggested by those skilled in the art, it is the intention of the inventors to embody within the patent warranted hereon all changes and modifications as reasonably and properly come within the scope of their contribution to the art.

We claim as our invention:

1. An arrangement for passive gas sampling of a breathing gas, comprising:
a tube adapted to receive a flow of breathing gas in an interior of the tube;
a measurement chamber in fluid communication with the interior of the tube; and
an element disposed in said interior of said tube for passively diverting a portion of said flow of breathing gas to said measurement chamber.

2. An arrangement as claimed in claim 1 wherein said element comprises a generally triangular element portion having a tip and a base, said tip being disposed closer to said measurement chamber than said base, said generally triangular element portion having a curved central axis proceeding between said base and said tip and having a cross-section orthogonal to said central axis that is flat at said base and is curved with a decreasing radius of curvature proceeding along said central axis between said base and said tip.

3. An arrangement as claimed in claim 2 wherein said element comprises a generally rectangular element portion disposed adjacent to said base of said generally triangular element portion.

4. An arrangement as claimed in claim 1 wherein said element has a T-shape with a stem and crosspiece, said stem being oriented toward said measurement chamber.

5. An arrangement for passive gas sampling of a breathing gas in a breathing system, comprising:
a housing;
a measurement chamber disposed in said housing;
said housing having a first connection adapted to receive an inspiration flow, a second connection adapted to discharge an expiration flow, and a third connection adapted to discharge the inspiration flow and to receive the expiration flow; and
said housing having a flow path therein configured so that said first connection and said third connection conduct the inspiration flow and the expiration flow along a substantially straight path toward said measurement chamber.
